(19) United States
(12) Patent Application Publication
(10) Pub. No.: US 2024/0071579 A1
(43) Pub. Date: Feb. 29, 2024

(54) SYSTEMS AND METHODS FOR DETERMINING CUSTOMIZED PAYMENT PLANS FOR MEDICAL BILLS

(71) Applicant: Capital One Services, LLC, McLean, VA (US)

(72) Inventors: Phoebe ATKINS, Midlothian, VA (US); Benjamin West POLLAK, Arlington, VA (US); Mia RODRIGUEZ, Broomfield, CO (US); Xiaoguang ZHU, New York, NY (US)

(21) Appl. No.: 17/893,537

(22) Filed: Aug. 23, 2022

(51) Int. Cl. G16H 10/60; G16H 50/20

(52) U.S. Cl. CPC G16H 10/60; G16H 50/20

(57) ABSTRACT

Systems and methods for active medical planning and customized budgeting may include a database that stores medical data, an interface, and a processor. The processor may be configured to receive and parse a medical document for medical procedure and related information, implement a learning algorithm to predict, based on the parsed medical and related information as well as the medical data, one or more medical procedures that the user is likely to undergo subsequent to any medical procedures parsed from the medical document, a cost associated with each predicted medical procedure, and a time horizon for each of the one or more predicted procedures, and train the predictive model with feedback from the user.

### FIELD OF THE DISCLOSURE

The present disclosure relates to systems and methods for active medical planning and customized budgeting.

### BACKGROUND

Medical billing can be confusing and unpredictable. A medical procedure often results in multiple bills from different places. For example, a minor surgery may result in bills from a doctor performing the surgery, a hospital, an anesthesiologist, and others. This can create significant issues in tracking paperwork and billing for patients, medical personnel, and insurance providers.

Similarly, health insurance plans can be confusing when trying to determine deductibles, negotiated rates, amounts paid towards out-of-pocket maximums, etc. The interaction between plan details and multiple bills for a single procedure, not to mention potential office visits pre- and post-operation or other medical procedures, can make it nearly impossible for a person to understand financial exposure and plan accordingly.

These and other deficiencies exist. Accordingly, there is a need for providing clarity with respect to medical procedures and billing, and also a need to provide assistance with budgeting to pay for those bills.

### SUMMARY OF THE DISCLOSURE

Embodiments of the present disclosure provide an active medical planning and customized budgeting system. The system may include a database, an interface, and a processor. The database may be configured to store medical procedures data, medical cost data, and historical user medical data. The processor may be configured to receive a user's medical document comprising one or more of a bill and an explanation of benefits, and including at least one medical procedure, apply natural language processing to the user's medical document, extract medical data from the user's medical document based on the natural language processing and a semantics engine to derive understanding and context from the user's medical document, the medical data comprising one or more medical procedures and for each of the one or more medical procedures, a plurality of a billing code for the procedure, a procedure cost, a procedure date, a responsible doctor, a hospital or practice associated with the procedure, a location of the procedure, and any explanation associated with the procedure, implement a predictive model, the implementation comprising, input the medical data extracted from the user's medical document into the predictive model, predict, based on the inputted medical data, historical user medical data, and medical procedures data, one or more medical procedures that the user is likely to undergo subsequent to each of the one or more medical procedures extracted from the user's medical document, a cost associated with each predicted medical procedure, and a time horizon for each of the one or more predicted procedures, and train the predictive model with feedback from the user, the feedback comprising one or more of explicit user feedback about the accuracy of the predictions and receipt of one or more subsequent medical documents evidencing the accuracy of the predicted medical procedures and predicted cost within the predicted timing for the one or more predicted medical procedures, display, via the customizable user interface, the one or more predicted additional medical procedures as well as a payment plan that provides the cost associated with each of the one or more predicted additional medical procedures.

Embodiments of the present disclosure provide a method for active medical planning and customized budgeting. The method may include receiving, via a processor, a user's medical document comprising one or more of a bill and an explanation of benefits, and including at least one medical procedure, applying, via the processor, natural language processing to the user's medical document, extracting, via the processor, medical data from the user's medical document based on the natural language processing and a semantics engine to derive understanding and context from the user's medical document, the medical data comprising one or more medical procedures and for each of the one or more medical procedures, a plurality of a billing code for the procedure, a procedure cost, a procedure date, a responsible doctor, a hospital or practice associated with the procedure, a location of the procedure, and any explanation associated with the procedure, implementing, via the processor, a predictive model, the implementation comprising: inputting the medical data extracted from the user's medical document into the predictive model, predicting, based on the inputted medical data, historical user medical data, and medical procedures data, one or more medical procedures that the user is likely to undergo subsequent to each of the one or more medical procedures extracted from the user's medical document, a cost associated with each predicted medical procedure, and a time horizon for each of the one or more predicted procedures; and training the predictive model with feedback from the user, the feedback comprising one or more of explicit user feedback about the accuracy of the predictions and receipt of one or more subsequent medical documents evidencing the accuracy of the predicted medical procedures and predicted cost within the predicted timing for the one or more predicted medical procedures, determining, via the processor, a cost associated with the one or more predicted additional medical procedures, displaying, via a customizable user interface, the one or more predicted additional medical procedures as well as a payment plan that provides the cost associated with each of the one or more predicted additional medical procedures.

Embodiments of the present disclosure provide a computer accessible non-transitory medium comprising computer-executable instructions that are executed on a processor and comprising the steps of: receiving, via a processor, a user's medical document comprising one or more of a bill and an explanation of benefits, and including at least one medical procedure, applying, via the processor, natural language processing to the user's medical document, extracting, via the processor, medical data from the user's medical document based on the natural language processing and a semantics engine to derive understanding and context from the user's medical document, the medical data comprising one or more medical procedures and for each of the one or more medical procedures, a plurality of a billing code for the procedure, a procedure cost, a procedure date, a responsible doctor, a hospital or practice associated with the procedure, a location of the procedure, and any explanation associated with the procedure, implementing, via the processor, a predictive model, the implementation comprising: inputting the medical data extracted from the user's medical document into the predictive model, predicting, based on the inputted medical data, historical user medical data, and medical procedures data, one or more medical procedures that the user is likely to undergo subsequent to each of the one or more medical procedures extracted from the user's medical document, a cost associated with each predicted medical procedure, and a time horizon for each of the one or more predicted procedures, and training the predictive model with feedback from the user, the feedback comprising one or more of explicit user feedback about the accuracy of the predictions and receipt of one or more subsequent medical documents evidencing the accuracy of the predicted medical procedures and predicted cost within the predicted timing for the one or more predicted medical procedures, displaying, via a customizable user interface, the one or more predicted additional medical procedures as well as a payment plan that provides the cost associated with each of the one or more predicted additional medical procedures.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Various embodiments of the present disclosure, together with further objects and advantages, may best be understood by reference to the following description taken in conjunction with the accompanying drawings.

FIG. 1 illustrates an active medical planning and customized budgeting system according to an exemplary embodiment.

FIG. 2 illustrates a sequence of operations for active medical planning according to an exemplary embodiment.

FIG. 3 illustrates a sequence of operations for active medical planning and customized budgeting according to an exemplary embodiment.

FIG. 4 is a schematic representation of a user device usable in embodiments of the invention.

FIG. 5 is a schematic representation of an application backend usable in embodiments of the invention.

FIG. 6 is a schematic representation of a machine learning algorithm module within the application backend, usable in embodiments of the invention.

FIG. 7 is a flow diagram illustrating a method of active medical planning and customized budgeting according to an embodiment of the invention.

### DETAILED DESCRIPTION

The following description of embodiments provides non-limiting representative examples referencing numerals to particularly describe features and teachings of different aspects of the invention. The embodiments described should be recognized as capable of implementation separately, or in combination, with other embodiments from the description of the embodiments. A person of ordinary skill in the art reviewing the description of embodiments should be able to learn and understand the different described aspects of the invention. The description of embodiments should facilitate understanding of the invention to such an extent that other implementations, not specifically covered but within the knowledge of a person of skill in the art having read the description of embodiments, would be understood to be consistent with an application of the invention.

The present invention provides systems and methods by which users may obtain insight into future medical procedures and bills based on an existing medical bill or explanation of benefits. The present invention also provides a customized financial plan for a user based on a cash flow analysis for the user. This may be accomplished by pulling relevant medical information from the provided medical document and then using that information to predict additional medical procedures, costs, and timing. The financial plan may be accomplished by analyzing a user's financial accounts to understand income vs expenses and then determine amounts available to pay for the predicted medical expenses. The systems and methods may also consider HSA and FSA accounts when proposing a financial plan.

The systems and methods for medical planning and customized budgeting provide customers with the ability to better understand their medical future and better plan for potential upcoming medical events within a known timeframe.

Further, the machine learning algorithm employed by the systems and methods for active medical planning and customized budgeting promotes system efficiency by reducing the demands on backend systems over time to improve the functioning of computers and conserve system resources.

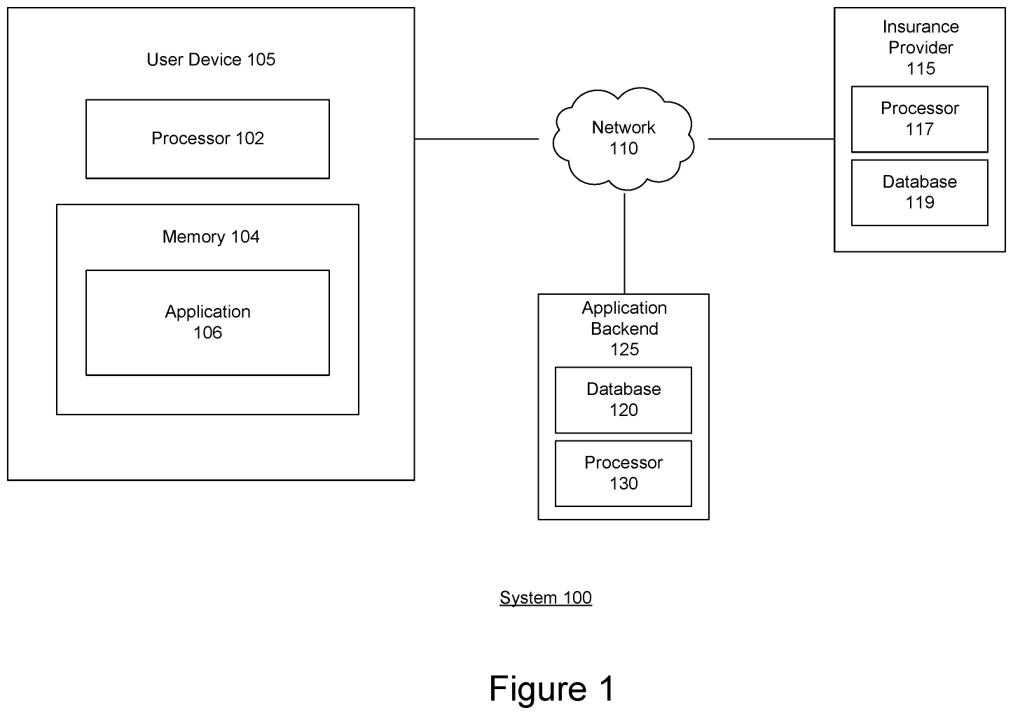

FIG. 1 illustrates an active web-based content filtering system 100. The system 100 may include a device 105, a network 110, an insurance provider server 115, and an application backend 125 with database 120 and processor 130. Although FIG. 1 illustrates single instances of components of system 100, system 100 may include any number of components.

System 100 may include a device 105. The device 105 may include one or more processors 102 and memory 104. Memory 104 may include one or more applications, such as application 106. The device 105 may be in data communication with any number of components of system 100. For example, the device 105 may transmit data via network 110 to database 120. Without limitation, the device 105 may be a network-enabled computer. As referred to herein, a network-enabled computer may include, but is not limited to a computer device, or communications device including, e.g., a server, a network appliance, a personal computer, a workstation, a phone, a handheld PC, a personal digital assistant, a contactless card, a thin client, a fat client, an Internet browser, a kiosk, a tablet, a terminal, an ATM, or other device. The device 105 also may be a mobile device; for example, a mobile device may include an iPhone, iPod, iPad from Apple® or any other mobile device running Apple's iOS® operating system, any device running Microsoft's Windows® Mobile operating system, any device running Google's Android® operating system, and/or any other smartphone, tablet, or like wearable mobile device.

The device 105 may include processing circuitry and may contain additional components, including processors, memories, error and parity/CRC checkers, data encoders, anticollision algorithms, controllers, command decoders, security primitives and tamper-proofing hardware, as necessary to perform the functions described herein. The device 105 may further include a display and input devices. The display may be any type of device for presenting visual information such as a computer monitor, a flat panel display, and a mobile device screen, including liquid crystal displays, light-emitting diode displays, plasma panels, and cathode ray tube displays. The input devices may include any device for entering information into the user's device that is available and supported by the user's device, such as a touch-screen, keyboard, mouse, cursor-control device, touch-screen, microphone, digital camera, video recorder or camcorder. These devices may be used to enter information and interact with the software and other devices described herein.

System 100 may include a network 110. In some examples, network 110 may be one or more of a wireless network, a wired network or any combination of wireless network and wired network, and may be configured to connect to any one of components of system 100. For example, the device 105 may be configured to connect to server 115 via network 110. In some examples, network 110 may include one or more of a fiber optics network, a passive optical network, a cable network, an Internet network, a satellite network, a wireless local area network (LAN), a Global System for Mobile Communication, a Personal Communication Service, a Personal Area Network, Wireless Application Protocol, Multimedia Messaging Service, Enhanced Messaging Service, Short Message Service, Time Division Multiplexing based systems, Code Division Multiple Access based systems, D-AMPS, Wi-Fi, Fixed Wireless Data, IEEE 802.11b, 802.15.1, 802.11n and 802.11g, Bluetooth, NFC, Radio Frequency Identification (RFID), Wi-Fi, and/or the like.

In addition, network 110 may include, without limitation, telephone lines, fiber optics, IEEE Ethernet 902.3, a wide area network, a wireless personal area network, a LAN, or a global network such as the Internet. In addition, network 110 may support an Internet network, a wireless communication network, a cellular network, or the like, or any combination thereof. Network 110 may further include one network, or any number of the exemplary types of networks mentioned above, operating as a stand-alone network or in cooperation with each other. Network 110 may utilize one or more protocols of one or more network elements to which they are communicatively coupled. Network 110 may translate to or from other protocols to one or more protocols of network devices. Although network 110 is depicted as a single network, it should be appreciated that according to one or more examples, network 110 may comprise a plurality of interconnected networks, such as, for example, the Internet, a service provider's network, a cable television network, corporate networks, such as credit card association networks, and home networks.

System 100 may include one or more insurance provider servers 115. In some examples, server 115 may include one or more processors 117 coupled to memory 119. Server 115 may be configured as a central system, server or platform to control and call various data at different times to execute a plurality of workflow actions. Server 115 may be configured to connect to application backend 125 via one or more networks 110. The server 115 may receive, for example, medical data requests from application backend 125 and may transmit data to application backend 125 in response to these requests.

In some examples, server 115 can be a dedicated server computer, such as bladed servers, or can be personal computers, laptop computers, notebook computers, palm top computers, network computers, mobile devices, wearable devices, or any processor-controlled device capable of supporting the system 100. While FIG. 1 illustrates a single server 115, it is understood that other embodiments can use multiple servers or multiple computer systems as necessary or desired to support the users and can also use back-up or redundant servers to prevent network downtime in the event of a failure of a particular server.

Server 115 may include an application in memory 119 comprising instructions for execution thereon. For example, the application may comprise instructions for execution on the server 115. The application may be in communication with any components of system 100. For example, server 115 may execute one or more applications that enable, for example, network and/or data communications with one or more components of system 100 and transmit and/or receive data. Without limitation, server 115 may be a network-enabled computer. As referred to herein, a network-enabled computer may include, but is not limited to a computer device, or communications device including, e.g., a server, a network appliance, a personal computer, a workstation, a phone, a handheld PC, a personal digital assistant, a contactless card, a thin client, a fat client, an Internet browser, or other device. Server 115 also may be a mobile device; for example, a mobile device may include an iPhone, iPod, iPad from Apple® or any other mobile device running Apple's iOS® operating system, any device running Microsoft's Windows® Mobile operating system, any device running Google's Android® operating system, and/or any other smartphone, tablet, or like wearable mobile device.

The server 115 may include processing circuitry and may contain additional components, including processors, memories, error and parity/CRC checkers, data encoders, anticollision algorithms, controllers, command decoders, security primitives and tamper-proofing hardware, as necessary to perform the functions described herein. The server 115 may further include a display and input devices. The display may be any type of device for presenting visual information such as a computer monitor, a flat panel display, and a mobile device screen, including liquid crystal displays, light-emitting diode displays, plasma panels, and cathode ray tube displays. The input devices may include any device for entering information into the user's device that is available and supported by the user's device, such as a touch-screen, keyboard, mouse, cursor-control device, touch-screen, microphone, digital camera, video recorder or camcorder. These devices may be used to enter information and interact with the software and other devices described herein.

System 100 may include one or more databases 120. The database 120 may comprise a relational database, a non-relational database, or other database implementations, and any combination thereof, including a plurality of relational databases and non-relational databases. In some examples, the database 120 may comprise a desktop database, a mobile database, or an in-memory database. Further, the database 120 may be hosted internally by any component of system 100, such as the device 105 or server 115, or the database 120 may be hosted externally to any component of the system 100, such as the device 105 or server 115, by a cloud-based platform, or in any storage device that is in data communication with the device 105 and server 115. In some examples, database 120 may be in data communication with any number of components of system 100. For example, the processor 102 in data communication with the application 106 may be configured to transmit one or more requests for the requested data from database 120 via network 110.

In some examples, exemplary procedures in accordance with the present disclosure described herein can be performed by a processing arrangement and/or a computing arrangement (e.g., computer hardware arrangement). Such processing/computing arrangement can be, for example entirely or a part of, or include, but not limited to, a computer/processor that can include, for example one or more microprocessors, and use instructions stored on a computer-accessible medium (e.g., RAM, ROM, hard drive, or other storage device). For example, a computer-accessible medium can be part of the memory of the device 105, server 115, and/or database 120, or other computer hardware arrangement.

In some examples, a computer-accessible medium (e.g., as described herein above, a storage device such as a hard disk, floppy disk, memory stick, CD-ROM, RAM, ROM, etc., or a collection thereof) can be provided (e.g., in communication with the processing arrangement). The computer-accessible medium can contain executable instructions thereon. In addition or alternatively, a storage arrangement can be provided separately from the computer-accessible medium, which can provide the instructions to the processing arrangement so as to configure the processing arrangement to execute certain exemplary procedures, processes, and methods, as described herein above, for example.

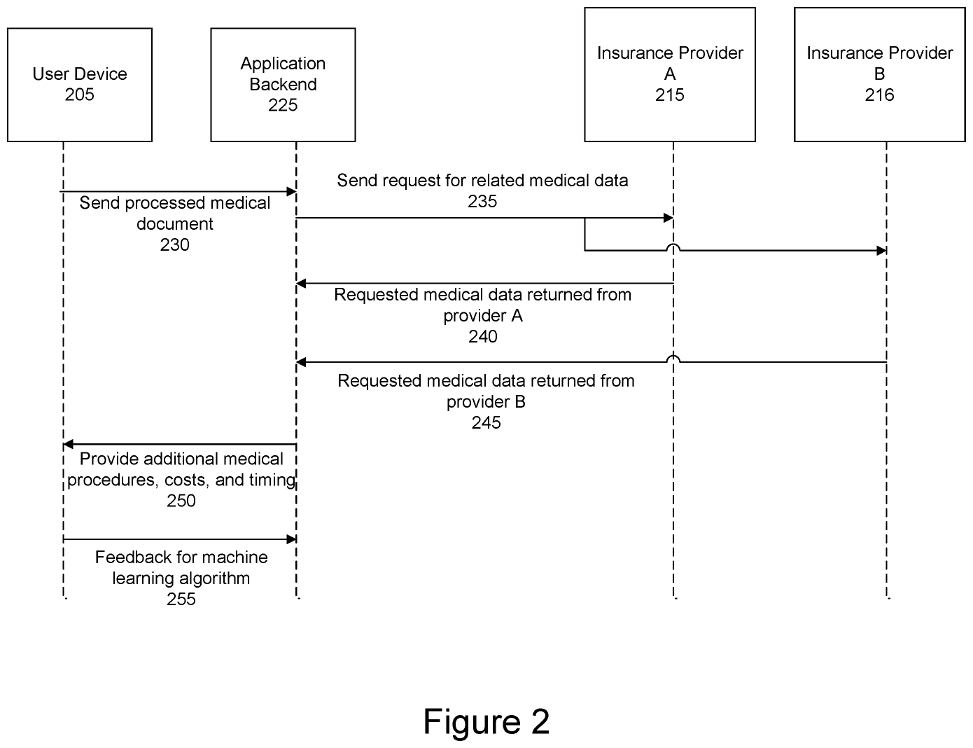

The sequence diagram of FIG. 2 illustrates an exemplary application of embodiments of the invention in conjunction with the system 100 of FIG. 1. In the scenario set forth in FIG. 2, a user device 205 is in communication with an application backend 225 and any number of insurance provider systems, illustrated in this figure by insurance provider A 215 and insurance provider B 216. User device 205 may a personal computer, smart phone, smart watch, or any other network enabled computing device. User device 205 may include memory, a network communication, an interactive user interface, and a processor capable of running one or more software applications. Application backend 225 may include a database capable of storing user medical transaction data, general medical information, and other data that may be relevant to locations, doctors, pricing, etc. of any medical procedure. Backend 225 may also include a processor capable of implementing machine learning algorithms to help predict medical procedures, including timing, costs, doctors, hospitals, and the like. Insurance providers A 215 and B 216 may constitute servers the include databases with procedure pricing by location, doctor, hospital, practice, etc. as well as coverage amounts for a given insurance policy, in network vs. out of network, negotiated rates, deductibles, out-of-pocket maximums, etc.

In the sequence of FIG. 2, a user, through user device 205, may scan, download, or otherwise access a medical document. The medical document may be a bill, an estimate, an explanation of benefits (“EOB”), or some other medical document presenting at least a medical procedure and a cost. The user may be prompted to load that medical document into an application running on user device 205. In response to that prompt, the user may load the medical document into the application. The application may then determine the content of the loaded medical document. This determination may be made by reading and understanding the medical document. The process for reading and understanding a webpage may involve natural language processing and a semantics engine capable of understanding combinations of structured and unstructured data. For example, the system may be capable of reading a sentence with a phrase that is recognized as a medical term. The system may further appreciate that the medical term constitutes a medical procedure and that this term is located proximate to a number. This number may be determined to be a universal medical code. The system may be able to associate the medical code with the medical procedure. The system may be further capable of finding pricing information associated with the medical code, the medical term, or both. The system may be capable of parsing the pricing information to understand if the pricing information constitutes a total cost, a deductible, a copay, a negotiated rate, a discount, or any combination of prices associated with the medical procedure and/or the medical code. The system may be able to infer this information by analyzing column or row headings for the various pricing data found on a given medical document. Other means of determining pricing and procedure information are possible based on contextual clues found during analysis of the medical document. The application may further be able to determine relevant dates for one or more medical procedures in the medical document as well as responsible physicians, practices, hospitals, etc. From this information, the application may infer a geographic location of the user. In another embodiment, the application may determine a user's geographic location from parsing the medical document.

The application may package all of the information that it pulls out of the medical document in a medical document file. At step 230, the application may send the medical document file to the application backend 225. In another embodiment, the application may send an image of the raw medical document to the application backend 225. In this instance, the parsing of the medical document, discussed above, may take place in the processor of the application backend 225.

Application backend 225 may store the medical document file in a database associated with the application backend 225. Application backend 225 may also send the received medical document file to a machine learning algorithm implemented by a processor associated with application backend 225. Application backend 225 may also store historical user medical transaction data, pooled user medical data, medical coding data, pricing information, provider information including doctors, practices, hospitals, etc. This data may be grouped in a variety of different ways, such as by specialty for a given procedure or by geographic location. For example, the system may receive medical data for a defined radius from the location identified in the medical document. The historical user medical transaction data may comprise a history of a user's medical transactions, which may provide insight into a user's medical history, as well as location, preferred doctors, practices, hospitals, etc. Pooled medical data may help provide insight as to what medical procedures may trigger other medical procedures, as well as where a doctor or practice may refer subsequent medical procedures, and/or if there are generally preferred doctors, hospitals, practices, etc. Additional data and relationships may be maintained. Medical coding data may be used to as a means for verifying the accuracy of the parsed medical document to make sure that the identified medical coding with medical terms matches the description of a medical code stored in the application backend 225. Pricing information may be gleaned from the pooled medical data by determining average procedure pricing, deductibles, negotiated rates, etc. on a per-provider basis. Based on the medical document file from user device 205, application backend 225 may send requests, at step 235, to one or more insurance providers, represented here by insurance provider A 215 and insurance provider B 216, for medical information relating to the medical procedure or procedures identified in the medical document file. The request for medical information may include details on a given medical procedure, one or more linked procedures, medical codes, billing codes, etc., information about various providers of the identified medical procedure, network status for the identified providers, and cost information including negotiated rates, deductibles, out-of-pocket maximums, etc. All of this information may be referenced with respect to a specific insurance plan owned by the user, or in the case of an insurance provider not issuing a user's policy, a similar plan serving as a proxy. At steps 240 and 245, the insurance providers A 215 and B 216 may return the requested medical data to application backend 225.

Application backend 225 may store the information form insurance providers A 215 and B 216. Application backend 225 may further aggregate all of the information from the medical document file, the insurance providers A 215 and B 216, and the application backend database, and use that data as an input to a machine learning algorithm running on the application backend 225. The machine learning algorithm may use the received data to predict one or more additional medical procedures that will likely follow the one or more medical procedures in the medical document loaded by the user. The machine learning algorithm may determine one or more relationships between a medical procedure listed in a medical document and any number of additional medical procedures. This relationship may be expressly stated in medical data from any of the insurance providers, in the historical medical data maintained in the application backend 225 database, or even in a user's medical transaction history. The relationship may also be inferred based on any number of relationships determined by the machine learning algorithm. For example, the system take a medical procedure in the medical document data and recognize a likelihood that any number of additional procedures will likely follow based on something in the user's medical transaction history that may increase the likelihood of a subsequent medical procedure. In some instances, the subsequent medical procedure or procedures may be standard, and therefore somewhat obvious. In other instances, the system might determine correlations and therefore relationships between seemingly unrelated medical conditions. The machine learning algorithm may hypothesize required subsequent medical procedures based on these correlations and may receive feedback on the hypothesis. The machine learning algorithm might further refine the predictions to understand the percent chance that a given subsequent medical procedure might be necessary, and this percent chance may be influenced by other data known to the machine learning algorithm. The machine learning algorithm might determine weights and rankings for subsequent medical procedures based on any number of factors including whether the subsequent procedures are explicitly stated in medical information—for example, a required follow-up after a given surgical procedure, or if a subsequent medical procedure is inferred or possible based on a given medical procedure—for example, getting a cast after an x-ray is not a certainty, but a possibility.

The machine learning algorithm may not only predict subsequent medical procedures, but also cost information and timing. Cost information may be predicted based on the machine learning algorithm's understanding of the likely subsequent medical procedures in light of a given medical insurance plan, as provided by the medical document file and cross referenced with one or more of the insurance providers. The predicted cost information may further account for in-network vs. out-of-network providers, a user's medical transaction history and what it suggests as far as user cost sensitivity vs. a preferred doctor, practice, hospital, etc., as well as general medical data and what it suggests as far as the likelihood that a person would have a predicted procedure completed by doctor A vs. doctor B, at hospital A vs. hospital B, etc. For example, if a predicted procedure is estimated to cost $1500 at doctor A and $2000 at doctor B, then the system may predict that the user will go to doctor A and therefore the predicted cost will be $1500. However, if the user's historical medical data indicates that the user has previously gone to doctor B and never visited doctor A, then the system may predict the cost will be $2000. In another example, if the system finds data to suggest that the user is cost sensitive, it may search additional providers to find one that costs less that either doctor A or B and predict that as the cost. In yet another example, if pooled medical data tends to indicate that for the geographic area surrounding the user, most people use doctor Z, regardless of price, then the system may predict that the user will go to doctor Z and may predict doctor Z's procedure cost. The machine learning algorithm may also consider whether doctors A, B, Z, or any other potential doctor is in-network or out-of-network when predicting a cost for a subsequent medical procedure.

The cost prediction from the machine learning algorithm may also take into account a user's deductible, out-of-pocket maximum, negotiated rates for a user's insurance plan, prior user medical expenses for the calendar year based on the user's historical medical transactions, as well as other factors. For example, if the machine learning algorithm predicts that a user will likely go to doctor A, and doctor A has a medical procedure cost of $1500, the machine learning algorithm may go further and not simply predict a $1500 cost. For instance, if a user has a policy with insurance provider A 215, and insurance company A has a negotiated rate of $800 for the procedure, then that may be the predicted cost. Furthermore, if the machine learning algorithm has insight into the user's deductible or copay, which may be 80%, for example, then the predicted cost may be 80% of $800, or $640. Further still, if the machine learning algorithm understands that the user's policy has a $3000 annual out-of-pocket maximum, and the user has already spent $2900 for medical care in the current year, then the algorithm may predict $100 for the subsequent medical procedure. This information may be gleaned from the medical document uploaded by the user or from the user's historical medical transaction data in the application backend database.

The machine learning algorithm may also predict a likely timing for each predicted subsequent medical procedure, relative to each other and also on an absolute basis. In this way, the machine learning algorithm may develop an overall picture of the user's medical future, including medical procedures, timings, and costs that will likely follow a detected medical procedure in an uploaded medical document. The predicted timing for each predicted medical procedure may be based on pooled historical medical data in the application backend database as well as data provided by any of the insurance providers A 215 and B 216.

At step 250, the application backend 225 may provide the predictions data including the predicted one or more additional medical procedures, the predicted cost for these procedures, and the relative and absolute timing for these procedures. The predicted procedures, costs, and timings may be presented to the user through the interactive user display of user device 205. This information may be useful in preparing, planning, budgeting, and facilitating the exchange of information between a user and a medical care provider. For example, if a user is made aware of potential upcoming medical procedures and when those procedures may occur, the user may be in a better position to request time off from work and get coverage for other commitments. Moreover, if a user is aware of upcoming medical costs, the user may create a budget to deal with those costs instead of being blindsided by multiple bills from various billers to a medical procedure (e.g., the hospital, the surgeon, the anesthesiologist, etc.). Also, if the user is made aware of a potential course of procedures, then that user may be better equipped to discuss the process with his or her medical provider and ask more and better questions.

The application running on the user device 205 may solicit feedback at or near the predicted procedure times to (1) understand if the predicted procedures were correct, and if not, what specifically was incorrect (e.g., no additional procedures were necessary, an alternate procedure was necessary, a similar procedure was necessary under a different medical procedure code, etc.), (2) if the predicted costs were accurate, and if not, what specifically was incorrect (e.g., the magnitude of cost was wrong and if so, was it because the procedure was done by a different doctor, at a different location, was out-of-network, etc., or the deductible, co-pay, maximum out of pocket, or negotiated rates were wrong, etc.), and (3) if the predicted timing of each predicted procedure was correct, and if not, what was the timing and specific reasons why. The application may also have access to ongoing medical records or documents for the user, and may parse these subsequent medical documents for the information without querying the user. In another embodiment, the application may use a subsequent medical document as the basis for querying a user for predictions feedback. In yet another embodiment, the user's subsequent medical data may be made available from insurance provider A 215 or insurance provider B 216. Context for that subsequent medical data may be provided by querying the user through the application on user device 205.

At step 255, the user feedback data may be sent to application backend 225. This data may be stored in the database and also sent the to machine learning algorithm as feedback to train, further refine, and improve the machine learning algorithm. The user feedback data may help train machine learning algorithm in a variety of different ways. For example, if a user indicates the accuracy of a predicted procedure, the machine learning algorithm may infer an accurate result. However, if the user indicates that a predicted procedure was not performed, then the machine learning algorithm might infer an incorrect prediction and may look into contextual data provided as to why the predicted medical procedure was not performed. If the user elected to skip the procedure even though it was recommended, the system may still infer a correct prediction. If no procedure was recommended by the doctor, then the system may take the feedback as an incorrect prediction. If a tangentially related medical procedure was performed instead, the system may infer a neutral result and seek to update weighting and rankings for potential subsequent medical procedure predictions. With respect to costs, similar inferences may be made. For example, if a cost prediction was more or less accurate, then the algorithm may infer a correct prediction. If the cost prediction was incorrect, then the reason for that inaccuracy must be considered and accounted for in the feedback analysis. In the event that the estimated costs from doctors, providers, or hospitals is incorrect, then adjustments may be made to pricing inputs. If there are issues with calculations of deductibles, out-of-pocket maximums and the like, then those portions of the analysis may be updated as well. If the cost prediction was wrong because the system predicted the wrong doctor, hospital, etc., then the weightings and rankings based on contextual data may be updated accordingly. The same sort of feedback also applies to predicted timing of predicted medical procedures. The machine learning algorithm may positively reinforce the algorithm upon correct predictions, and may analyze data and adjust analysis where predictions are inaccurate. The foregoing are examples of how the user feedback data may be used by the machine learning algorithm and are not meant to be exhaustive.

With continued feedback and training of the machine learning algorithm over time, the machine learning algorithm may not only become more accurate, but also more efficient since there is less computing necessary as the machine learning algorithm becomes more confident of procedures, costs and timing predictions. Thus, not only is the accuracy of the predictions improved over time, but the functioning of the computer is also improved over time as the machine learning algorithm is trained.

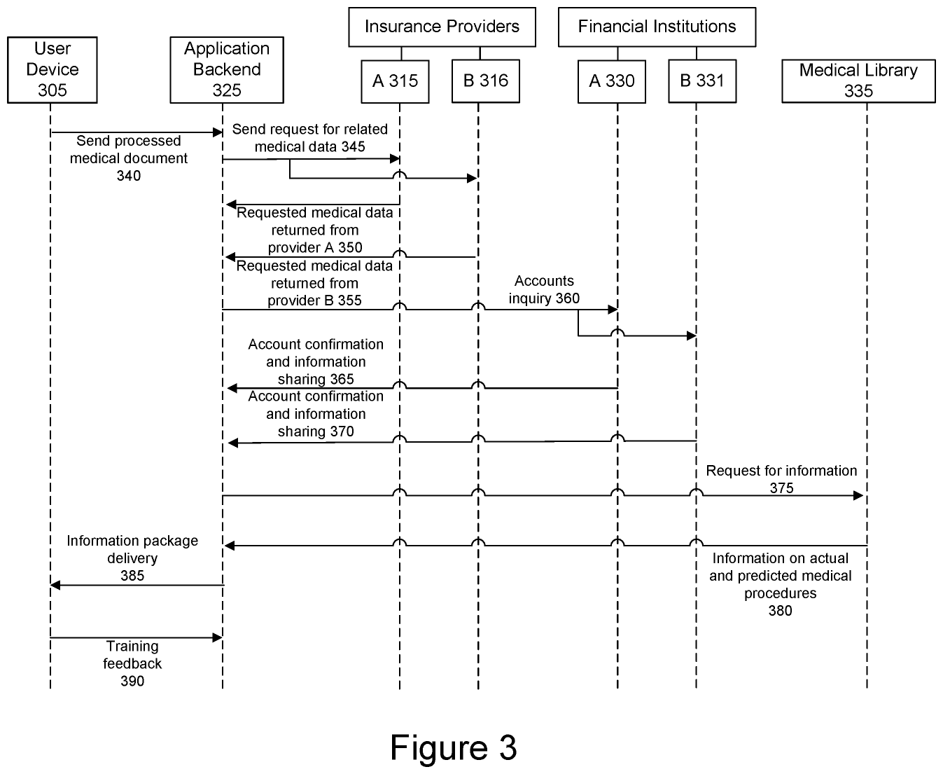

In some embodiments, it may be desirable to link the system to one or more user accounts for added functionality and benefit to the user. It may also be desirable to link the system to one or more medical libraries to obtain and integrate additional information. The sequence diagram of FIG. 3 illustrates an exemplary scenario in which the backend engages with one or more financial institution's systems, as well as a medical library.

A user may interact with an application on a user device 305. The application may be communicatively coupled with an application backend 325 for the application via a communication interface on user device 305. The application may be an application associated with a financial entity or some other entity. The application may require user login and may require authorization to integrate with one or more user financial accounts as well as track or allow access to one or more repositories for the user's medical records, including with one or more health insurance providers. A user may be prompted to enter a medical document into the application. The medical document may be a bill, and EOB, or any other medical document listing a medical procedure for the user. The application may then process the medical document to pull relevant information that may be sent to application backend 325 in step 340. Processing the medical document may entail reading and understanding the medical document in order to determine and extract the relevant content. The process for reading and understanding a webpage may involve natural language processing and a semantics engine capable of understanding combinations of structured and unstructured data. For example, the system may be capable of reading a sentence and detecting a phrase within that sentence that is a medical term. The system may determine that the medical term is, or is related to, a medical procedure. The system may then search the document proximate to the detected medical term for any sort of numbers. Any detected numbers are then analyzed to determine if those numbers are associated with a medical billing code, or a cost. Contextual clues may help in this analysis. For example, a “$” preceding or near the number may infer the number is a cost, whereas a “#” near the number may infer an ID number. Other contextual clues may be used such as row or column headings, or any other information that tends to give context to the number detected near the medical term. The system may be capable of parsing any pricing information to understand if the pricing information constitutes a total cost, a deductible, a copay, a negotiated rate, a discount, or any combination of prices associated with the medical procedure and/or the medical code. The system may be able to infer this information by analyzing column or row headings for the various pricing data found on a given medical document. Other means of determining pricing and procedure information may be possible based on contextual clues found during analysis of the medical document. The analysis may also look for dates of service and billing dates associated with the medical procedure(s), as well as other potentially pertinent information such as the biller's identity, the provider of the medical procedure (e.g., doctor, practice, hospital, etc.). From this information, the application may infer a geographic location of the user. In another embodiment, the application may determine a user's geographic location from parsing the medical document to find a user's address.

The application may package all of the information that it pulls out of the medical document in a medical document file. At step 340, the application may send the medical document file to the application backend 325. In another embodiment, the application may send an image of the raw medical document, or a partially processed version of the medical document to the application backend 325. In this instance, the parsing of the medical document, discussed above, may take place in the processor of the application backend 325.

Application backend 325 may store the medical document file in a database associated with the application backend 325. Application backend 325 may also send the received medical document file to a machine learning algorithm implemented by a processor associated with application backend 325. Application backend 325 may also store historical user medical transaction data, pooled user medical data, medical coding data, pricing information, provider information including doctors, practices, hospitals, etc. This data may be grouped in a variety of different ways, such as by specialty for a given procedure or by geographic location. The historical user medical transaction data may comprise a history of a user's medical transactions, which may provide insight into a user's medical history, as well as location, preferred doctors, practices, hospitals, etc. Pooled medical data may help provide insight as to what medical procedures may trigger other medical procedures, as well as where a doctor or practice may refer subsequent medical procedures, and/or if there are generally preferred doctors, hospitals, practices, etc. Additional data and relationships may be maintained. Medical coding data may be used to as a means for verifying the accuracy of the parsed medical document to make sure that the identified medical coding with medical terms matches the description of a medical code stored in the application backend 225. Pricing information may be gleaned from the pooled medical data by determining average procedure pricing, deductibles, negotiated rates, etc. on a per-provider basis. Based on the medical document file from user device 305, application backend 325 may send requests, at step 345, to one or more insurance providers, represented here by insurance provider A 315 and insurance provider B 316, for medical information relating to the medical procedure or procedures identified in the medical document file. The request for medical information may include details on a given medical procedure, one or more linked procedures, medical codes, billing codes, etc., information about various providers of the identified medical procedure, network status for the identified providers, and cost information including negotiated rates, deductibles, out-of-pocket maximums, etc. All of this information may be referenced with respect to a specific health insurance plan owned by the user, or in the case of an insurance provider not issuing a user's policy, a similar plan serving as a proxy. At steps 350 and 355, the insurance providers A 315 and B 316 may return the requested medical data to application backend 325. Application backend 325 may store the information form insurance providers A 315 and B 316.

The application backend 325 may seek financial information relating to the user. In some embodiments, the application backend is associated with a financial institution. In this scenario, the application backend 325 may pull financial statements, transactions, records, and/or other financial data for one or more of the user's accounts held with the financial institution. Regardless of whether the application and application backend 325 are associated with a financial institution, at step 360 application backend 325 may query one or more financial institutions, represented in FIG. 3 by financial institution A 330 and financial institution B 331, to see if the user holds any accounts with financial institution A 330 or financial institution B 331. The query may also request financial data for the user to the extent that the user holds any accounts with the financial institutions. The requested financial data may include financial statements, transactions, records, and/or other financial data. The data may include recurring direct deposits that may represent income as well as one-off deposits and recurring and one-off payments and expenditures out of an account. At steps 365 and 370, financial institutions A 330 and B 331 respectively may provide a response to the query from the application backend 325. The responses may include confirmation of the existence of lack of one or more user accounts as well as the requested financial data, when applicable. In one example, a user may hold one savings account and one checking account with financial institution A 330, and no accounts with financial institution B 331. In this scenario, the request would seek a response from both financial institutions. The response from financial institution B 331 would be that the user holds no accounts. The response from financial institution A 330 would include a listing of accounts as well as the financial data for each account. The access to the user's financial accounts and financial data may be authorized and set-up by the user, through the application on the user device 305. The user's financial accounts may also include flexible spending accounts (“FSA”) and health savings accounts (HSA).

Application backend 325 may aggregate all of the information from the medical document file, the insurance providers A 315 and B 316, the application backend database, and financial institutions A 330 and B 331, and use that data as an input to a machine learning algorithm running on the application backend 325. The machine learning algorithm may use the received data to predict one or more additional medical procedures that will likely follow the one or more medical procedures in the medical document loaded by the user. The machine learning algorithm may determine one or more relationships between a medical procedure listed in a medical document and any number of additional medical procedures. This relationship may be expressly stated in medical data from any of the insurance providers, in the historical medical data maintained in the application backend 325 database, or even in a user's medical transaction history. The relationship may also be inferred based on any number of relationships determined by the machine learning algorithm. For example, the system take a medical procedure in the medical document data and recognize a likelihood that any number of additional procedures will likely follow based on something in the user's medical transaction history that may increase the likelihood of a subsequent medical procedure. In some instances, the subsequent medical procedure or procedures may be standard, and therefore be somewhat obvious. In other instances, the system may determine correlations and therefore relationships between seemingly unrelated medical conditions. The machine learning algorithm may hypothesize required subsequent medical procedures based on these correlations and may receive feedback on the hypothesis. The machine learning algorithm might further refine the predictions to understand a likelihood or percent chance that a given subsequent medical procedure might be necessary, and this likelihood may be influenced by other data known to the machine learning algorithm. The machine learning algorithm might determine weights and rankings for subsequent medical procedures based on any number of factors including whether the subsequent procedures are explicitly stated in medical information—for example, a required follow-up after a given surgical procedure, or if a subsequent medical procedure is inferred or possible based on a given medical procedure—for example, getting a cast after an x-ray is not a certainty, but a possibility.

The machine learning algorithm may not only predict subsequent medical procedures, but also cost information and timing. Cost information may be predicted based on the machine learning algorithm's understanding of the likely subsequent medical procedures in light of a given medical insurance plan, as provided by the medical document file and cross referenced with one or more of the insurance providers. The predicted cost information may further account for in-network vs. out-of-network providers, a user's medical transaction history and what it suggests as far as user cost sensitivity vs. a preferred doctor, practice, hospital, etc., as well as general medical data and what it suggests as far as the likelihood that a person would have a predicted procedure completed by doctor A vs. doctor B, at hospital A vs. hospital B, etc. For example, if a predicted procedure is estimated to cost $1500 at doctor A and $2000 at doctor B, then the system may predict that the user will go to doctor A and therefore the predicted cost will be $1500. However, if the user's historical medical data indicates that the user has previously gone to doctor B and never visited doctor A, then the system may predict the cost will be $2000. In another example, if the system finds data to suggest that the user is cost sensitive, it may search additional providers to find one that costs less that either doctor A or B and predict that as the cost. In yet another example, if pooled medical data tends to indicate that for the geographic area surrounding the user, most people use doctor Z, regardless of price, then the system may predict that the user will go to doctor Z and may predict doctor Z's procedure cost. The machine learning algorithm may also consider whether doctors A, B, Z, or any other potential doctor is in-network or out-of-network when predicting a cost for a subsequent medical procedure.

The cost prediction from the machine learning algorithm may also take into account a user's deductible, out-of-pocket maximum, negotiated rates for a user's insurance plan, prior user medical expenses for the calendar year based on the user's historical medical transactions, as well as other factors. For example, if the machine learning algorithm predicts that a user will likely go to doctor A, and doctor A has a medical procedure cost of $1500, the machine learning algorithm may go further and not simply predict a $1500 cost. For instance, if a user has a policy with insurance provider A 215, and insurance company A has a negotiated rate of $800 for the procedure, then that may be the predicted cost. Furthermore, if the machine learning algorithm has insight into the user's deductible or copay, which may be 80%, for example, then the predicted cost may be 80% of $800, or $640. Further still, if the machine learning algorithm understands that the user's policy has a $3000 annual out-of-pocket maximum, and the user has already spent $2900 for medical care in the current year, then the algorithm may predict $100 for the subsequent medical procedure. This information may be gleaned from the medical document uploaded by the user or from the user's historical medical transaction data in the application backend database.

The machine learning algorithm may also predict a likely timing for each predicted subsequent medical procedure, relative to each other and also on an absolute basis. In this way, the machine learning algorithm may develop an overall picture of the user's medical future, including medical procedures, timings, and costs that will likely follow a detected medical procedure in an uploaded medical document. The predicted timing for each predicted medical procedure may be based on pooled historical medical data in the application backend database as well as data provided by any of the insurance providers A 315 and B 316.

The machine learning algorithm may take the overall picture of the user's medical future and reference the user's financial data to determine a budget and a plan to pay the predicted future expenses. For instance, the machine learning algorithm may receive financial account statements and recognize recurring deposits over time for the same or similar amounts. The algorithm may infer these deposits to be income paid from an employer or from some type of work performed by the user. The machine learning algorithm may further analyze the user's financial data to get a sense for cash flow on a periodic basis, often on a monthly basis. This analyzed cash flow may include not just perceived income, but also recurring fixed monthly expenses and expenditures such as rent, mortgage, car payment, insurance, utilities, etc. and variable recurring monthly expenses such as gas, tolls, food, etc. It may also include an analysis of the user's common spend for other categories of goods and services such as entertainment, clothing, etc. In the event the user has linked FSAs or HSAs with the application, then the algorithm may further consider cash flow in each of those accounts. Once the machine learning algorithm has a sense of cash flow, it may understand what money may be available to pay for the predicted costs of the predicted medical procedures. In one embodiment, the machine learning algorithm may place a preference on paying the predicted medical expenses with medical expense accounts such as FSAs and HSAs first before paying with any other common user account. This may especially be true in the case of a FSA since that money may be lost at the end of a calendar year if the money is unused. In another embodiment, the algorithm may appreciate that a user may prefer to not use funds from a HSA because the user may be utilizing that account as a tax deferred means to save money toward retirement. In one embodiment, the user may specify this preference in the Application. In another embodiment, the application may prompt the user to provide these types of preferences. In still another embodiment, the machine learning algorithm may infer the user's preference based on an analysis of the cash flow in the HSA and the investment strategy within the HSA. For example, if the HSA has regular periodic deposits into the account and no withdraws, then the algorithm may infer that the user does not want to use the HSA to pay for current or medical expenses, or those arising in the near future. Also, if the HSA is invested for aggressive growth, then the algorithm may infer that the user does not intend to use the HSA funds in then near-term. The machine learning algorithm may develop a proposed payment plan for the predicted medical expenses in light of the timeframe or time horizon for those predicted medical procedures and the user's cash flow through the linked accounts. This payment plan may be optimized for various goals. For example, one goal may be to maximize after-tax liquidity, while another goal may be to maximized tax deferred money in one or more accounts. Other goals may be considered when developing a proposed payment plan.

At step 375, the application backend 325 may send a request for medical information to one or more medical library 335. In one embodiment, medical library 335 may include online medical information databases. The request for medial information may relate to one or more of the predicted medical procedures and may include potential side effects, recovery times, risks, success rates, and any other information that a user may want to know about a predicted medical procedure. The request for this medical information may include application backend 325 accessing the information on one or more websites and scraping the information from those websites. At step 380, the medical information relating to the predicted medical procedures may be provided to the application backend 325. As discussed, in one embodiment, this step may also be accomplished through scraping the information from one of more websites containing the relevant medical information.

At step 385, the application backend 325 may package some or all of the data—the predicted additional medical procedures, the predicted costs of the predicted medical procedures, the predicted timing for the predicted medical procedures, the proposed budget and payment plan for the predicted medical procedure costs, as well as the medical information pertaining to the predicted medical procedures. The packaged data may be presented to the user through the interactive user display of user device 305. The information may be presented in any logical way. For example, on a first page, the predicted medical procedures may be displayed on a predicted timeline. A basis for the predictions may be provided in any number of ways such as placing the information directly on the page, having that information embedded behind the predicted medical procedures on the timeline, through drop-down boxes, etc. The cost information may be included on that page, or presented separately on a subsequent page. The cost information may be presented along with the proposed budget and payment plan. A basis for the proposed budget and payment plan may also be provided. The medical information based on the predicted medical procedures may be provided on a separate page and may further prove links to additional information. The packaged data may be useful in preparing, planning, budgeting, and facilitating the exchange of information between a user and a medical care provider. For example, if a user is made aware of potential upcoming medical procedures and when those procedures may occur, the user may be in a better position to request time off from work and get coverage for other commitments. Moreover, if a user is aware of upcoming medical costs, then based on the proposed budget, the user may avoid other expenses. Also, if the user is made aware of a potential course of procedures, then that user may be better equipped to discuss the process with his or her medical provider and ask more and better questions.

The application running on the user device 305 may solicit feedback at or near the predicted procedure timing to (1) understand if the predicted procedures were correct, and if not, what specifically was incorrect (e.g., no additional procedures were necessary, an alternate procedure was necessary, a similar procedure was necessary under a different medical procedure code, etc.), (2) if the predicted costs were accurate, and if not, what specifically was incorrect (e.g., the magnitude of cost was wrong and if so, was it because the procedure was done by a different doctor, at a different location, was out-of-network, etc., or the deductible, co-pay, maximum out of pocket, or negotiated rates were wrong, etc.), and (3) if the predicted timing of each predicted procedure was correct, and if not, what was the timing and specific reasons why. The application may also have access to ongoing medical records or documents for the user, and may parse these subsequent medical documents for the information without querying the user. In another embodiment, the application may use a subsequent medical document as the basis for querying a user for predictions feedback. In yet another embodiment, the user's subsequent medical data may be made available from insurance provider A 315 or insurance provider B 316. Context for that subsequent medical data may be provided by querying the user through the application on user device 305.

At step 390, the user feedback data may be sent to application backend 325. This data may be stored in the database and also sent to the machine learning algorithm as feedback to train, further refine, and improve the machine learning algorithm. The user feedback data may help train the machine learning algorithm in a variety of different ways. For example, if a user indicates the accuracy of a predicted procedure, the machine learning algorithm may infer an accurate result. However, if the user indicates that a predicted procedure was not performed, then the machine learning algorithm might infer an incorrect prediction and may look into contextual data provided as to why the predicted medical procedure was not performed. If the user elected to skip the procedure even though it was recommended, the system may still infer a correct prediction. If no procedure was recommended by the doctor, then the system may take the feedback as an incorrect prediction. If a tangentially related medical procedure was performed instead, the system may infer a neutral result and seek to update weighting and rankings for potential subsequent medical procedure predictions. With respect to costs, similar inferences may be made. For example, if a cost prediction was more or less accurate, then the algorithm may infer a correct prediction. If the cost prediction was incorrect, then the reason for that inaccuracy must be considered and accounted for in the feedback analysis. In the event that the estimated costs from doctors, providers, or hospitals is incorrect, then adjustments may be made to pricing inputs. If there are issues with calculations of deductibles, out-of-pocket maximums and the like, then those portions of the analysis may be updated as well. If the cost prediction was wrong because the system predicted the wrong doctor, hospital, etc., then the weightings and rankings based on contextual data may be updated accordingly. The same sort of feedback also applies to predicted timing of predicted medical procedures. The machine learning algorithm may positively reinforce the algorithm upon correct predictions, and may analyze data and adjust analysis where predictions are inaccurate. The foregoing are examples of how the user feedback data may be used by the machine learning algorithm and are not meant to be exhaustive.

With continued feedback and training of the machine learning algorithm over time, the machine learning algorithm may not only become more accurate, but also more efficient since there is less computing necessary as the machine learning algorithm becomes more confident of procedures, costs and timing predictions. Thus, not only is the accuracy of the predictions improved over time, but the functioning of the computer is also improved over time as the machine learning algorithm is trained.

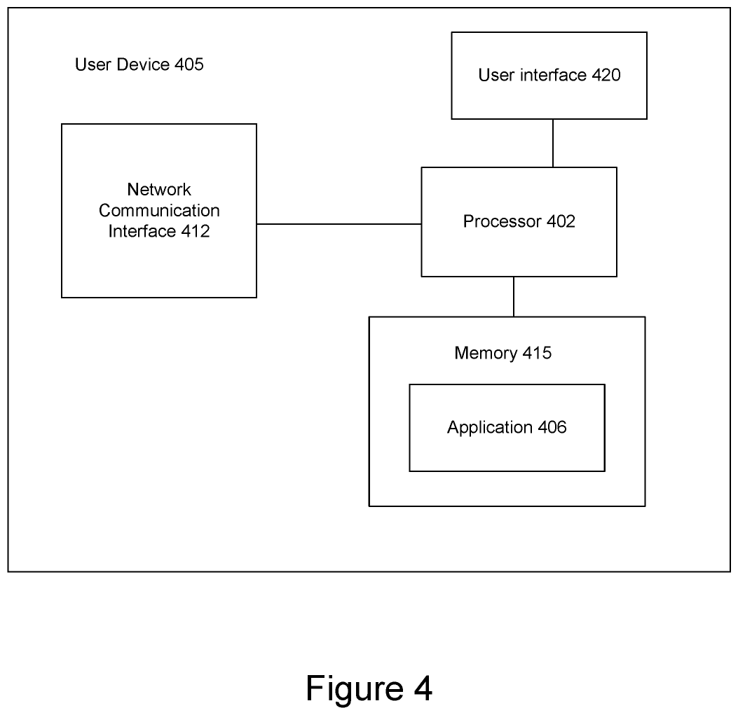

With reference to FIG. 4, user device 405 may be any computer device or communications device including a server, a network appliance, a personal computer (PC), a workstation, and a mobile interface device such as a smart phone, smart watch, smart pad, handheld PC, or personal digital assistant (PDA). In a particular embodiment illustrated in FIG. 4, user device 405 includes an on-board processor 402 in communication with memory 415, a user interface 420, and a network communication interface 412. The processor 402 may include a microprocessor and associated processing circuitry, and can contain additional components, including processors, memories, error and parity/CRC checkers, data encoders, anticollision algorithms, controllers, command decoders, security primitives and tamper-proofing hardware, as necessary to perform the functions described herein. The memory 415 can be a read-only memory, write-once read-multiple memory or read/write memory, e.g., RAM, ROM and EEPROM, and the user device 405 can include one or more of these memories.

The user interface 420 of the device 405 includes a user input mechanism, which can be any device for entering information and instructions into the user device 405, such as a touch-screen, keyboard, mouse, cursor-control device, microphone, stylus, or digital camera. The user interface 420 may also include a display, which can be any type of device for presenting visual information such as a computer monitor, a flat panel display, and a mobile device screen, including liquid crystal displays, light-emitting diode displays, plasma panels, and cathode ray tube displays.

The network communication interface 412 is configured to establish and support wired and/or wireless data communication capability for connecting the user device 405 to the network 110 or other communication network. The network communication interface 412 can also be configured to support communication with a short-range wireless communication interface, such as Bluetooth.

In embodiments of the invention, the memory 415 may have stored therein application 406 usable by the processor 402. Application 406 may include instructions usable by the processor 402 to register a user, receive medical documents, process those medical documents, link the application to one or more health insurance accounts and to one or more financial accounts.

As a user launches application 406, in one embodiment, the application 406 may prompt the user to provide preferences for payment of medical bills (e.g., use a FSA account first, then common accounts, followed by HSA accounts). The application 406 may prompt the user to upload a medical document. In another embodiment, the application may follow a link, provided by the user, to a medical document. Application 406 may be configured to process the medical document by pulling out relevant information including any medical procedures, medical codes associated with the medical procedures, costs for the procedures, provider information including practice, doctor, hospital, etc., dates, and locations. This process is explained in detail with respect to FIGS. 2 and 3. The application may package this relevant information and send it to an application backend over a network, via the network communication interface 412.

Application 406 may present, via user interface 420, an information package to the user based on the uploaded medical document. The information package may include one or more predicted additional medical procedures, based on the one or more medical procedures detected in the medical document, predicted costs for each of the predicted medical procedures, and predicted timing for each of those procedures. In some embodiments, the information package may include a proposed budget and payment plan based on an analysis of the user's finances. In some embodiments, the information package may also include medical information related to the predicted medical procedures. The medical information may come from medical databases.

Application 406 may query the user regarding the accuracy of the predictions provided in the information package. The prompts for feedback may coincide with the predicted timing to better capture information at the correct time. In some embodiments, application 406 may determine the accuracy of the predictions by accessing medical records, documents, etc. Application 406 may send this feedback information to the application backend for the purpose of training a machine learning algorithm that made the predictions in the user information package.

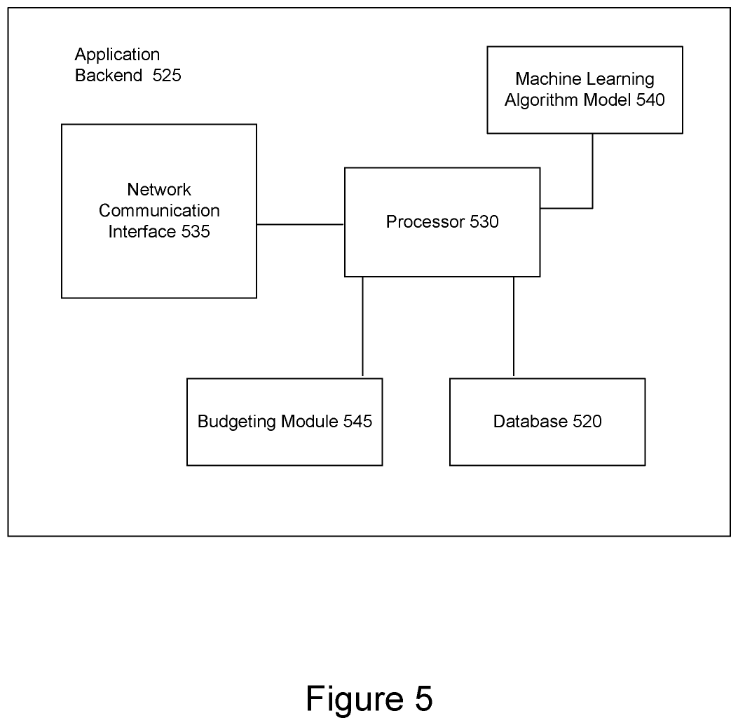

With reference to FIG. 5, application backend 525 may be a server such as a dedicated server computer, such as bladed servers, or personal computer, laptop computer, notebook computer, palm top computer, network computer, or any processor-controlled device capable of supporting the system 100. While FIG. 5 illustrates an application backend 525 that may be a single server, it is understood that other embodiments can use multiple servers or multiple computer systems as necessary or desired to support the users and can also use back-up or redundant servers to prevent network downtime in the event of a failure of a particular server. In a particular embodiment illustrated in FIG. 5, application backend 525 includes a processor 530 in communication with a database 520, a network communication interface 535, a budgeting module 545, and a machine learning algorithm module 540. The processor 530 may include a microprocessor and associated processing circuitry, and can contain additional components, including processors, memories, error and parity/CRC checkers, data encoders, anticollision algorithms, controllers, command decoders, security primitives and tamper-proofing hardware, as necessary to perform the functions described herein. The database 520 my comprise memory and can be a read-only memory, write-once read-multiple memory or read/write memory, e.g., RAM, ROM and EEPROM, and the user device 405 can include one or more of these memories.

The network communication interface 535 is configured to establish and support wired and/or wireless data communication capability for connecting the user device 405 to the network 110 or other communication network. The network communication interface 535 can also be configured to support communication with a short-range wireless communication interface, such as Bluetooth.

In embodiments of the invention, the processor 530 may receive a medical data document, via network communication interface 535, containing information about a medical procedure for a user. This medical data document may include the name of one or more medical procedures for a user, it may include a billing code for the procedure as well as cost information. The cost information may include a procedure cost, a negotiated rate, a billed amount, a copay and/or a deductible amount, an amount owed by user, etc. The document may also include other information related to the medical procedure such as a procedure date, a responsible doctor, a hospital or practice where the procedure was administered, an address for the doctor/practice/hospital/user, etc. In some embodiments, processor 530 may receive a medical document such as a bill or a EOB and may parse that document to extract the medical data. Processor 530 may store that medical data in database 520 and also pass the information onto machine learning algorithm module 540 as inputs. Processor 530 may also receive pooled user medical data from a pool of users and/or from authorized health insurance providers. This information may also be stored in database 520. Application backend 525 may also receive user financial data from user financial records generated in connection with a financial entity owning application backend 525, and/or from any number of other financial institutions. The financial data may include money coming into one or more accounts and money going out of the one or more accounts. Processor 530 may store this data in database 520. The processor 530 may also send the data to budgeting module 545 where this data may be analyzed. Budgeting module 545 may analyze the cash flow in each of the user's financial accounts. This analysis may include determining regular periodic deposits to an account. These regular deposits may be in the form of direct deposits and may be interpreted as income. The budgeting module 545 may differentiate these deposits from other non-periodic deposits, or even other periodic deposits that may not represent income from a job. The budgeting module 545 may also determine regular withdraws or drafts on an account and infer that these are part of fixed expenses. Module 545 may also determine monthly payments for credit cards or other revolving lines of credit and further determine an average monthly spend on each of these determined accounts. In this way, the budgeting module may develop a financial picture for the user. This financial picture may include a monthly income vs expense analysis, free cash flow, potential for savings, etc. The budgeting module may provide this financial picture to the machine learning algorithm module 540, via processor 530, and may also store the financial picture in database 520.

Machine learning algorithm module 540 may receive the medical data, pooled user data, and financial data as inputs to help predict future medical procedures that a user will likely require, a cost for those procedures, a timeframe for those procedures, and a proposed personalized budget to cover the costs of those procedures. Machine learning algorithm module 540 may also receive feedback on these predicted items to further train and refine the machine learning algorithm, and to further reduce loads on a computer or server executing the machine learning algorithm.

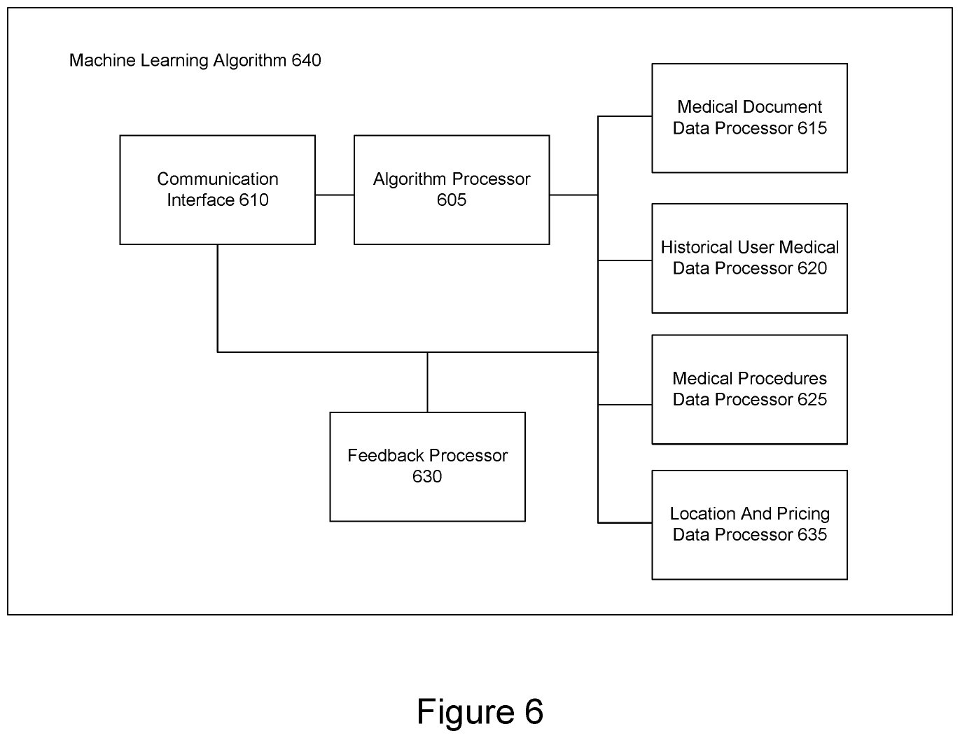

With reference to FIG. 6, machine learning algorithm 640 may be part of application backend 525. Machine learning algorithm 640 may include a communication interface 610 as well as an algorithm processor 605 coupled to a plurality of additional processors including medical document data processor 615, historical user medical data processor 620, medical procedures data processor 625, location and pricing data processor 635, and feedback processor 630. The processors of FIG. 6 may include microprocessors and associated processing circuitry, and can contain additional components, including processors, memories, error and parity/CRC checkers, data encoders, anticollision algorithms, controllers, command decoders, security primitives and tamper-proofing hardware, as necessary to perform the functions described herein. It should be appreciated that while FIG. 6 depicts multiple discrete processors, the machine learning algorithm may be accomplished by any number of processors including a single processor.

Machine learning algorithm 640 may receive a number of inputs from the application backend through communication interface 610. These inputs may include a user's medical data extracted from a medical document. This medical data may include the names of one or more medical procedures performed on a user as well as related information such as a medical or billing code for each medical procedure, a procedure date, who administered the procedure, where the procedure was administered, and cost information. “Who administered the procedure” might be a doctor, a practice, a hospital, etc. “Where the procedure was administered” might be a doctor's office, a practice, a hospital, etc. Cost information may include standard rates each procedure, one or more negotiated rates—these rates may be negotiated by one or more health insurance provider, user deductible or copay information, out of pocket maximum information including a maximum amount and an amount paid towards that maximum, amount due, etc. The inputs may also include pooled historical medical data among a group of people. This data may include information regarding medical procedures, pricing, timing, etc. This data may help provide insight as to what medical procedures may trigger other medical procedures, as well as where a doctor or practice may refer subsequent medical procedures, and/or if there are generally preferred doctors, hospitals, practices, etc. The inputs may further include medical procedures data stored in database 520 or received from one or more health insurance provider databases. This information may include medical coding information for one or more medical procedures, linked medical procedures, general information about a medical procedure, pricing information for a medical procedure, explanations of one or more health insurance plans, etc. Inputs to the machine learning algorithm 640 may also include pricing information by location, practice, hospital, doctor, etc. This information may be obtained from a health insurance provider database, or may be stored locally in database 520.

The medical data from the user's medical document may be utilized by medical document data processor 615 to help predict future medical procedures, pricing, and timing. For instance, data may go into medical document data processor 615 and be analyzed for context and relational data. This relational data may allow the machine learning algorithm 640 to more fully understand the user's probable medical situation and therefore form the basis for linking the medical procedures in the medical document to additional medical procedures.

The historical user medical data processor 620 may rely on the identified medical procedure(s) in the medical document as well as other user historical medical data and pooled medical data to get a better understanding of what is likely to follow an identified medical procedure in the medical document. For example, if a user has lower back x-rays and has a medical history of back problems, then the system may infer that a MRI is likely to follow. The system may consider pooled medical data. That pooled medical data may provide statistics as to the likelihood that someone in the user's situation would have a Mill. The pooled medical data may consider any of a multitude of factors such as age, weight, etc., and then further refine the statistics for predicting the likelihood of the user needing a MRI. Processor 620 may further weight the factors. For example, if the need for a Mill is more strongly correlated with age than height, then the age factor may be weighed more heavily in predicting the likelihood that a user will need a Mill. Similarly, if a user's past medical history is more strongly correlated than any factor from pooled data, then that might be weighed more heavily. The historical user medical data processor may further analyze pooled data to get an idea of what procedures may be necessary after a MM. For example, in one scenario, the data may indicate that of the users comprising the pooled data, 25% of people have no further procedure following a lower back MM, 50% of people have a microdiscectomy, 15% undergo a spinal fusion, and 10% undergo a different medical procedure. The historical user medical data processor 620 may utilize that data to help understand the likelihood that the user may need a subsequent medical procedure following a MM, and what that procedure might be. The data may also be considered in light of various factors such as age, sex, height, weight, etc. Processor 620 may find other relationships in the data with respect to a target medical procedure in a user's medical document.

Historical user medical data processor 620 may also help in the prediction of cost and timing for potential additional user medical procedures. For example, if a user has medical history that covers the potential additional medical procedures, then that data may be useful in predicting the cost of the potential additional medical procedures. Also, information about the user's health insurance plan will help provide context for the cost to the user for an additional medical procedure, regardless of the standard or uninsured cost. Pooled medical data may be helpful in predicting costs because that data may include prices actually paid by users for the potential additional medical procedures. This data may also be considered by the location and pricing data processor 635. From a pricing perspective, if the data shows that users tend to be price sensitive for a given procedure, then the system may weigh the lowest cost options more heavily. In some circumstances, the data may indicate that the majority of users go to a particular doctor for a given medical procedure regardless of price. In those circumstances, the processor 620 may weigh the cost of that doctor more heavily than either more expensive or less expensive options. In this way, the processor 620 may be able to get a better idea of the potential cost of a medical procedure.

Location and pricing data processor 635 may consider the prices charged by doctors, hospitals, etc. offering each potential medical procedure. For example, if machine learning algorithm 640 understands there is a high likelihood that a user might go to doctor A for a potential medical procedure, processor 635 may pull cost information for doctor A for that procedure. The system may then cross reference that doctor with the user's medical plan and determine a negotiated rate for the procedure as well as the user's copay, deductible, etc. In some instance, algorithm 640 may consider multiple potential doctors for a given procedure and then compare pricing among each of those doctors and weigh the results based on any of a plethora of potential factors. For example, if doctor B is more cost effective than doctor A, but doctor B does not have availability within the user's timing window, then that doctor and his or her pricing may be ignored in the analysis. Location may also be considered. If a user shows a propensity for staying within a certain geographic area, then doctors outside of that area may be weighted less, even if their prices would otherwise weigh them more highly. This information may also be considered within the context of the pooled medical data and where people are generally willing to travel for a given procedure. For example, if a procedure is more serious, then a user may be more willing to travel further than for a more routine and minor procedure.

Medical procedures data processor 625 may use a medical procedure identified in a user's medical document as a starting point for referencing additional medical data from a database of medical information. The database of medical information may be supplied by one or more health insurance providers and may provide insights as to known relationships between various medical procedures, conditions, etc. This data may consider linked or related medical procedures, linked or related medical or billing codes, or other relationship data farmed by one or more health insurance providers. Processor 625 may use this information to help predict the likelihood of any additional user medical procedures.

Algorithm processor 605 may receive all of the information from processors 615, 620, 625, and 635 and determine the most probable additional medical procedures, pricing, and timing. In some embodiments, algorithm processor 605 may also consider a user's financial picture from budgeting module 545 to develop a proposed payment plan to cover the predicted medical expenses. In another embodiment, the machine learning algorithm provides the predicted medical costs to budgeting module 545, and module 545 may develop a budget and payment plan accordingly.

Feedback processor 630 may receive user feedback regarding the predicted additional medical procedures, cost, and timing. This feedback may be solicited from the user at or near the predicted procedure timing to (1) understand if the predicted procedures were correct, and if not, what specifically was incorrect (e.g., no additional procedures were necessary, an alternate procedure was necessary, a similar procedure was necessary under a different medical procedure code, etc.), (2) if the predicted costs were accurate, and if not, what specifically was incorrect (e.g., the magnitude of cost was wrong and if so, was it because the procedure was done by a different doctor, at a different location, was out-of-network, etc., or the deductible, co-pay, maximum out of pocket, or negotiated rates were wrong, etc.), and (3) if the predicted timing of each predicted procedure was correct, and if not, what was the timing and specific reasons why. Feedback may also be in the form of ongoing user medical records or documents. Feedback processor 630 may use the feedback data to positively reinforce where predictions were correct, and why. Similarly, processor 630 may use the feedback to understand where and why mistakes were made in the predictions. The feedback processor may determine weights for positive reinforcement and corrections for mistakes, and then send this feedback information into processor 605 to further refine and train machine learning algorithm 640.

The predictive models described herein may utilize various neural networks, such as convolutional neural networks (“CNNs”) or recurrent neural networks (“RNNs”), to generate the exemplary models. A CNN may include one or more convolutional layers (e.g., often with a subsampling step) and then followed by one or more fully connected layers as in a standard multilayer neural network. CNNs may utilize local connections, and may have tied weights followed by some form of pooling which may result in translation invariant features.

A RNN is a class of artificial neural network where connections between nodes form a directed graph along a sequence. This facilitates the determination of temporal dynamic behavior for a time sequence. Unlike feedforward neural networks, RNNs may use their internal state (e.g., memory) to process sequences of inputs. A RNN may generally refer to two broad classes of networks with a similar general structure, where one is finite impulse and the other is infinite impulse. Both classes of networks exhibit temporal dynamic behavior. A finite impulse recurrent network may be, or may include, a directed acyclic graph that may be unrolled and replaced with a strictly feedforward neural network, while an infinite impulse recurrent network may be, or may include, a directed cyclic graph that may not be unrolled. Both finite impulse and infinite impulse recurrent networks may have additional stored state, and the storage may be under the direct control of the neural network. The storage may also be replaced by another network or graph, which may incorporate time delays or may have feedback loops. Such controlled states may be referred to as gated state or gated memory, and may be part of long short-term memory networks (LSTMs) and gated recurrent units

RNNs may be similar to a network of neuron-like nodes organized into successive “layers,” each node in a given layer being connected with a directed e.g., (one-way) connection to every other node in the next successive layer. Each node (e.g., neuron) may have a time-varying real-valued activation. Each connection (e.g., synapse) may have a modifiable real-valued weight. Nodes may either be (i) input nodes (e.g., receiving data from outside the network), (ii) output nodes (e.g., yielding results), or (iii) hidden nodes (e.g., that may modify the data en route from input to output). RNNs may accept an input vector x and give an output vector y. However, the output vectors are based not only by the input just provided in, but also on the entire history of inputs that have been provided in in the past.

For supervised learning in discrete time settings, sequences of real-valued input vectors may arrive at the input nodes, one vector at a time. At any given time step, each non-input unit may compute its current activation (e.g., result) as a nonlinear function of the weighted sum of the activations of all units that connect to it. Supervisor-given target activations may be supplied for some output units at certain time steps. For example, if the input sequence is a speech signal corresponding to a spoken digit, the final target output at the end of the sequence may be a label classifying the digit. In reinforcement learning settings, no teacher provides target signals. Instead, a fitness function, or reward function, may be used to evaluate the RNNs performance, which may influence its input stream through output units connected to actuators that may affect the environment. Each sequence may produce an error as the sum of the deviations of all target signals from the corresponding activations computed by the network. For a training set of numerous sequences, the total error may be the sum of the errors of all individual sequences.

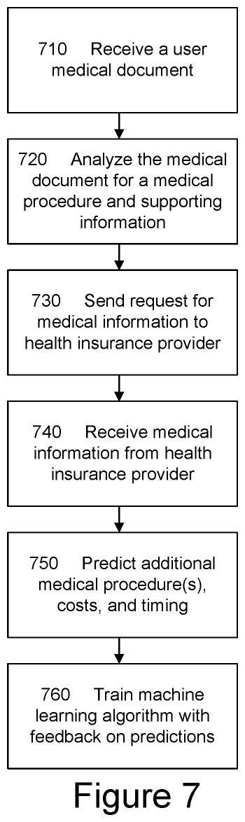

FIG. 7 illustrates an exemplary method for implementing an active medical planning and budgeting framework system according to an embodiment of the invention. The actions of the method depicted in FIG. 7 may be carried out by a user device in conjunction with an application backend and may result in the delivery and display of a predicted future medical plan with budgeting and payment suggestions. At step 710, an application running on a user device may receive a medical document uploaded by a user. The medical document may be a medical services bill, and EOB, or any other medical document. At step 720, the application may parse and analyze the medical document. The parsing and analysis of the medical document may include determining the content of the loaded medical document by reading and understanding the medical document. The process for reading and understanding a webpage may involve natural language processing and a semantics engine capable of understanding combinations of structured and unstructured data. For example, the application may be capable of reading a sentence with a phrase that is recognized as a medical term. The application may further appreciate that the medical term constitutes a medical procedure and that this term is located proximate to a number. This number may be determined to be a universal medical code. The application may be able to associate the medical code with the medical procedure. The application may be further capable of finding pricing information associated with the medical code, the medical term, or both. The application may be capable of parsing the pricing information to understand if the pricing information constitutes a total cost, a deductible, a copay, a negotiated rate, a discount, or any combination of prices associated with the medical procedure and/or the medical code. The system may be able to infer this information by analyzing column or row headings for the various pricing data found on a given medical document. Other means of determining pricing and procedure information are possible based on contextual clues found during analysis of the medical document. The application may further be able to determine relevant dates for one or more medical procedures in the medical document as well as responsible physicians, practices, hospitals, etc. From this information, the application may infer a geographic location of the user. In another embodiment, the application may determine a user's geographic location from parsing the medical document. The application may pull out this medical information (e.g., medical procedure(s), medical/billing codes, cost information, timing information, doctor/practice/hospital information, location information, etc.) and package it in a medical data file. This medical data file may be stored in a database associated with the application backend

At step 730, an application backend may request medical data from one or more health insurance providers. The request may be based on the medical information packaged in the medical data file. For instance, if the medical data file indicates that the user underwent procedure A, then the medical data request may seek information relevant to procedure A, such as general cost information, related procedures including likely subsequent procedures, and general medical knowledge about the medical procedure or a condition related to the medical procedure. If the medical data file includes a specific doctor, then the information request may include cost information for that specific doctor. Other related data may be requested. The information request may also include a verification of user coverage by the health insurance provider as well as details of the policy, such as deductibles, copays, out of pocket maximums, in-network providers, etc. At step 740, the application backend may receive a response from each of the health insurance providers. The response may include a verification of whether the user holds a policy with the health insurance provider, as well as the other requested information. This information may be stored in a database associated with the application backend.

At step 750, a machine learning algorithm running on the application backend may predict one or more medical procedures that are likely to be subsequent to the medical procedure(s) in the medical data file. The machine learning algorithm may also predict a cost associated with each of those predicted medical procedures and the likely timing for each of the predicted medical procedures. Further details of the machine learning algorithm are described with respect to figures, 2, 3, and 6. In some embodiments, the application backend stores, or has access to, user financial account records. The application backend may analyze those financial account records to determine a user's cash flow and further determine the user's ability to pay for the predicted medical costs. The application backend may develop a financial plan for paying the predicted costs for the predicted medical procedures.

The application backend may then pass the predicted medical procedures, costs, timing, and proposed financial plan to the user, via the user device. Periodically, and especially within time windows coinciding with the predicted timing of the additional medical procedures, the application may solicit feedback from the user to ascertain the accuracy of each prediction. This may also include the soliciting feedback for the proposed financial plan. At step 760, this feedback may be used as an input to the machine learning algorithm to train and refine the function of the machine learning algorithm. The user feedback data may help train machine learning algorithm in a variety of different ways. For example, if a user indicates the accuracy of a predicted procedure, the machine learning algorithm may infer an accurate result. However, if the user indicates that a predicted procedure was not performed, then the machine learning algorithm might infer an incorrect prediction and may look into contextual data provided as to why the predicted medical procedure was not performed. If the user elected to skip the procedure even though it was recommended, the system may still infer a correct prediction. If no procedure was recommended by the doctor, then the system may take the feedback as an incorrect prediction. If a tangentially related medical procedure was performed instead, the system may infer a neutral result and seek to update weighting and rankings for potential subsequent medical procedure predictions. With respect to costs, similar inferences may be made. For example, if a cost prediction was more or less accurate, then the algorithm may infer a correct prediction. If the cost prediction was incorrect, then the reason for that inaccuracy must be considered and accounted for in the feedback analysis. In the event that the estimated costs from doctors, providers, or hospitals is incorrect, then adjustments may be made to pricing inputs. If there are issues with calculations of deductibles, out-of-pocket maximums and the like, then those portions of the analysis may be updated as well. If the cost prediction was wrong because the system predicted the wrong doctor, hospital, etc., then the weightings and rankings based on contextual data may be updated accordingly. The same sort of feedback also applies to predicted timing of predicted medical procedures. The machine learning algorithm may positively reinforce the algorithm upon correct predictions, and may analyze data and adjust analysis where predictions are inaccurate. The foregoing are examples of how the user feedback data may be used by the machine learning algorithm and are not meant to be exhaustive.

The foregoing are examples of how the user interaction data may be used as feedback to the machine learning algorithm and are not meant to be exhaustive. With continued feedback and training of the machine learning algorithm over time, the machine learning algorithm may not only become more accurate, but also more efficient since there is less computing necessary as the machine learning algorithm becomes more confident of procedures, costs and timing predictions. Thus, not only is the accuracy of the predictions improved over time, but the functioning of the computer is also improved over time as the machine learning algorithm is trained.

It is further noted that the systems and methods described herein may be tangibly embodied in one or more physical media, such as, but not limited to, a compact disc (CD), a digital versatile disc (DVD), a floppy disk, a hard drive, read only memory (ROM), random access memory (RAM), as well as other physical media capable of data storage. For example, data storage may include random access memory (RAM) and read only memory (ROM), which may be configured to access and store data and information and computer program instructions. Data storage may also include storage media or other suitable type of memory (e.g., such as, for example, RAM, ROM, programmable read-only memory (PROM), erasable programmable read-only memory (EPROM), electrically erasable programmable read-only memory (EEPROM), magnetic disks, optical disks, floppy disks, hard disks, removable cartridges, flash drives, and any type of tangible and non-transitory storage medium), where the files that comprise an operating system, application programs including, for example, web browser application, email application and/or other applications, and data files may be stored. The data storage of the network-enabled computer systems may include electronic information, files, and documents stored in various ways, including, for example, a flat file, indexed file, hierarchical database, relational database, such as a database created and maintained with software from, for example, Oracle® Corporation, Microsoft® Excel file, Microsoft® Access file, a solid state storage device, which may include a flash array, a hybrid array, or a server-side product, enterprise storage, which may include online or cloud storage, or any other storage mechanism. Moreover, the figures illustrate various components (e.g., servers, computers, processors, etc.) separately. The functions described as being performed at various components may be performed at other components, and the various components may be combined or separated. Other modifications also may be made.

It will be readily understood by those persons skilled in the art that the present invention is susceptible to broad utility and application. Many embodiments and adaptations of the present invention other than those herein described, as well as many variations, modifications and equivalent arrangements, will be apparent from or reasonably suggested by the present invention and foregoing description thereof, without departing from the substance or scope of the invention.

We claim:

1. An active medical planning and customized budgeting system, comprising:
a database that stores medical procedures data, medical cost data, and historical user medical data;
a user interface; and
a processor configured to:
receive a medical document associated with a user, the medical document identifying at least one medical procedure and comprising at least one selected from a group of a bill and an explanation of benefits;
apply natural language processing to the medical document;
extract medical data from the medical document based on the natural language processing and a semantics engine to derive understanding and context from the medical document, the medical data comprising one or more medical procedures and for each of the one or more medical procedures, at least two selected from a group of a billing code for the procedure, a procedure cost, a procedure date, a responsible doctor, a hospital or practice associated with the procedure, a location of the procedure, and any explanation associated with the procedure;
implement a predictive model, the implementation comprising:
input the medical data extracted from the medical document into the predictive model;
predict, based on the inputted medical data, historical user medical data, and medical procedures data, one or more medical procedures that the user is likely to undergo subsequent to each of the one or more medical procedures extracted from the medical document, a cost associated with each predicted medical procedure, and a time horizon for each of the one or more predicted procedures; and
train the predictive model with feedback from the user, the feedback comprising at least one selected from a group of explicit user feedback about the accuracy of the predictions and receipt of one or more subsequent medical documents evidencing the accuracy of the predicted medical procedures and predicted cost within the predicted timing for the one or more predicted medical procedures; and
display, by the customizable user interface, the one or more predicted medical procedures and a payment plan that provides the cost associated with each of the one or more predicted medical procedures.

2. The system of claim 1, wherein predicting a cost associated with the one or more predicted medical procedures comprises receiving cost data for a plurality of care providers within a defined radius of the location of service in the medical document and providing one or more cost options to the user.

3. The system of claim 1, wherein the predictive model is used to determine where the user will likely receive the one or more predicted medical procedures based on the medical cost data and historical user medical data, further wherein the historical user medical data reveals one or more preferred care provider.

4. The system of claim 1, wherein the predictive model is trained with feedback from a user provider selection based on cost or location.

5. The system of claim 1, wherein the processor is further configured to access one or more user financial accounts to determine income and expenditures as well as amounts already paid against the one or more medical procedures in the medical document.

6. The system of claim 5, wherein the processor is further configured to determine a budget for paying the predicted cost of each predicted medical procedure based on an analysis of the one or more user financial accounts.

7. The system of claim 6, wherein the prediction is integrated with one or more of a user HSA and a user FSA.

8. The system of claim 7, wherein the payment plan is designed to achieve a stated goal and includes a suggested monthly payment amount from the one or more user financial accounts as well as a monthly payment amount from the one or more of the HSA and FSA, the stated goal comprising one of maximizing after tax liquidity and maximizing tax deferred money.

9. The system of claim 1, wherein the processor is further configured to reference cost data on one or more insurance company databases.

10. The system of claim 1, wherein the processor is further configured to access one or more medical information libraries to collect information about the one or more predicted medical procedures, and display the collected information to the user, via the customizable user interface, along with the one or more predicted medical procedures and the payment plan.

11. A method for active medical planning and customized budgeting, comprising:
receiving, via a processor, a medical document associated with a user, the medical document identifying at least one medical procedure and comprising at least one selected from a group of a bill and an explanation of benefits;
applying, via the processor, natural language processing to the medical document;
extracting, via the processor, medical data from the medical document based on the natural language processing and a semantics engine to derive understanding and context from the medical document, the medical data comprising one or more medical procedures and for each of the one or more medical procedures, at least two selected from a group of a billing code for the procedure, a procedure cost, a procedure date, a responsible doctor, a hospital or practice associated with the procedure, a location of the procedure, and any explanation associated with the procedure;
implementing, via the processor, a predictive model, the implementation comprising:
inputting the medical data extracted from the medical document into the predictive model;
predicting, based on the inputted medical data, historical user medical data, and medical procedures data, one or more medical procedures that the user is likely to undergo subsequent to each of the one or more medical procedures extracted from the medical document, a cost associated with each predicted medical procedure, and a time horizon for each of the one or more predicted procedures; and
training the predictive model with feedback from the user, the feedback comprising at least one selected from a group of explicit user feedback about the accuracy of the predictions and receipt of one or more subsequent medical documents evidencing the accuracy of the predicted medical procedures and predicted cost within the predicted timing for the one or more predicted medical procedures; and
displaying, via a customizable user interface, the one or more predicted medical procedures and a payment plan that provides the cost associated with each of the one or more predicted medical procedures.

12. The method of claim 11, wherein predicting a cost associated with the one or more predicted medical procedures comprises receiving cost data for a plurality of care providers within a defined radius of the location of service in the medical document and providing one or more cost options to the user.

13. The method of claim 11, wherein the predictive model is used to determine where the user will likely receive the one or more predicted medical procedures based on the medical cost data and historical user medical data, further wherein the historical user medical data reveals one or more preferred care provider.

14. The method of claim 11, wherein the predictive model is trained with feedback from a user's provider selection based on cost or location.

15. The method of claim 11, wherein the processor is further configured to access one or more user financial accounts to determine income and expenditures as well as amounts already paid against the one or more medical procedures in the medical document.

16. The method of claim 15, wherein the processor is further configured to determine a budget for paying the predicted cost of each predicted medical procedure based on an analysis of the one or more user financial accounts.

17. The method of claim 16, wherein the prediction is integrated with one or more of a user HSA and a user FSA.

18. The method of claim 17, wherein the payment plan is designed to achieve a stated goal and includes a suggested monthly payment amount from the one or more user financial accounts as well as a monthly payment amount from the one or more of the HSA and FSA, the stated goal comprising one of maximizing after tax liquidity and maximizing tax deferred money.

19. The method of claim 11, wherein the processor is further configured to reference cost data on one or more insurance company databases.

20. A computer-accessible non-transitory medium comprising computer-executable instructions that, when executed by at least one processor, configure the processor to perform procedures comprising:
receiving a medical document associated with a user, the medical document identifying at least one medical procedure and comprising at least one selected from a group of a bill and an explanation of benefits;
applying natural language processing to the user's medical document;
extracting, medical data from the medical document based on the natural language processing and a semantics engine to derive understanding and context from the medical document, the medical data comprising one or more medical procedures and for each of the one or more medical procedures, at least two selected from a group of a billing code for the procedure, a procedure cost, a procedure date, a responsible doctor, a hospital or practice associated with the procedure, a location of the procedure, and any explanation associated with the procedure;
implementing a predictive model, the implementation comprising:
inputting the medical data extracted from the user's medical document into the predictive model;
predicting, based on the inputted medical data, historical user medical data, and medical procedures data, one or more medical procedures that the user is likely to undergo subsequent to each of the one or more medical procedures extracted from the medical document, a cost associated with each predicted medical procedure, and a time horizon for each of the one or more predicted procedures; and
training the predictive model with feedback from the user, the feedback comprising at least one selected from a group of explicit user feedback about the accuracy of the predictions and receipt of one or more subsequent medical documents evidencing the accuracy of the predicted medical procedures and predicted cost within the predicted timing for the one or more predicted medical procedures; and
displaying, via a customizable user interface, the one or more predicted medical procedures and a payment plan that provides the cost associated with each of the one or more predicted medical procedures.
